(19) United States
(12) Patent Application Publication
(10) Pub. No.: US 2026/0053566 A1
(43) Pub. Date: Feb. 26, 2026

(54) Method and system for the improvement of a virtual surgical procedure

(71) Applicants: Politecnico di Milano, Milano MI (IT); Fondazione IRCCS Istituto Neurologico Carlo Besta, Milano MI (IT); Artiness S.r.l., Milano MI (IT)

(72) Inventors: Alberto Cesare Luigi REDAELLI, Milano MI (IT); Maria Chiara PALUMBO, Milano MI (IT); Simone SAITTA, Milano MI (IT); Maria Chiara SBARRA, San Giorgio Ionico TA (IT); Eleonora TURCONI, Seregno MB (IT); Villiam DALLOLIO, Lecco LC (IT); Marco Paolo SCHIARITI, Milano MI (IT); Paolo FERROLI, Milano MI (IT)

(21) Appl. No.: 19/102,976

(22) PCT Filed: Jul. 26, 2023
(86) PCT No.: PCT/IB2023/057583

§ 371 Date: Feb. 11, 2025

(30) Foreign Application Priority Data
Aug. 12, 2022 (IT) 102022000017319

(51) Int. Cl. A61B 34/10; A61B 90/00; A61B 90/50; G06N 3/0464

(52) U.S. Cl. CPC A61B 34/10; A61B 90/36; A61B 90/50; G06N 3/0464; A61B 2034/105; A61B 2034/107; A61B 2090/365; A61B 2090/372; A61B 2090/502

(57) ABSTRACT

System for the improvement of a virtual cranial surgical procedure, and relative method comprising the steps of: identifying a target of said surgical procedure; generating by means of an artificial intelligence algorithm a three-dimensional holographic model of the portion of the patient's skull by processing preoperative clinical images; identifying the target of the surgical procedure in the three-dimensional holographic model; acquiring in real time a depth image of the real environment by means of a holographic device; identifying by means of the holographic device a pointer movable by the user in the holographic model; identifying an entry point of the surgical procedure selected by the user by means of said pointer on the three-dimensional holographic model displayed on the display; calculating a surgical trajectory connecting the entry point to the target in the three-dimensional holographic model; displaying by means of the display of the visor the surgical trajectory together with the three-dimensional holographic model in overlay to the real environment.

### FIELD OF APPLICATION

The present invention relates to a method and system for the improvement of a virtual surgical procedure, and in particular for the improvement of a cranial surgical procedure.

### DESCRIPTION OF THE PRIOR ART

It is known in the art to realize a method for improving a surgical procedure by providing a three-dimensional model of a patient's organ based on preoperative image data of the patient's organ, an example of which is shown in document US 2018/263704 A1. In particular, the method of document US 2018/263704 A1 comprises the step of identifying the data relating to the position of at least one target treatment anatomy of the patient with respect to a second position of an auxiliary target anatomy of the patient on the basis of an analysis of the three-dimensional model of the patient's organ. This method subsequently comprises selecting a puncture position based on the position data identified for the two target anatomies and displaying, by means of an augmented reality device, a virtual organ corresponding to the three-dimensional model that overlays the real-world environment, visually indicating the position of the selected puncture.

In addition, document US 2021/330416 A1 shows a method for improving a surgical procedure substantially analogous to the method of document US 2018/263704 A1.

### Problem of the Prior Art

In the known technique, and in particular in document US 2018/263704 A1, the method makes it possible to identify the puncture position as a function of two target anatomies of the patient's organ. However, the known method of document US 2018/263704 A1 cannot take into account the anatomical and clinical factors of the actual patient as the calculation of the puncture position is performed exclusively on the three-dimensional model of the organ. Therefore, there is a risk that the puncture position calculated by means of the known method is not the one optimal for the patient. In fact, given the complexity of the surgeon's choice of the puncture position, this operation is always evaluated and decided in first person by the surgeon during the operative step and as a function of the conditions of the actual patient.

### SUMMARY OF THE INVENTION

Aim of the invention in question is to realize a method and a system that allow to overcome the drawbacks of the known technique, favouring the improvement of a cranial surgical procedure.

A further aim of the invention is to provide a method and a system for increasing the success rate in craniotomy, craniectomy or drainage catheter insertion operations by exploiting the display of holograms related to the patient-specific 3D model through a head-mounted display and overlay of these models to the patient.

The specified technical task and objects are substantially achieved by a method and a system comprising the technical characteristics as set out in one or more of the accompanying claims.

### ADVANTAGES OF THE INVENTION

Thanks to an embodiment, it is possible to obtain a method and a system that allow to reduce the times of preparation of a surgical procedure in order to intervene in an emergency, for example by providing further useful information for the surgeon in the selection of the surgical instruments, in the choice of the entry point of the procedure and in the choice of the position of the patient for surgery.

Thanks to an embodiment, it is possible to realize a method and a system which are very quick in the processing, which allow to improve the chances of success and surgery performances.

Thanks to an embodiment, it is possible to realize a method and a system that allow to calculate a surgical trajectory specific for the patient and for the real conditions, starting from an entry point manually identified by the surgeon, which takes into account fundamental variables such as, for example, the internal condition of the tissues, so as to avoid any obstacles in the surgical trajectory.

Thanks to an embodiment, it is possible to realize a method and a system that take advantage of mixed reality to favour the perfectioning of a surgical procedure.

### BRIEF DESCRIPTION OF THE DRAWINGS

The characteristics and the advantages of the present invention will be apparent from the following detailed description of a possible practical embodiment, illustrated by way of non-limiting example in the set of drawings, in which:
FIG. 1 schematically shows a system according to the present invention in an example of application of the method of the invention,
FIG. 2 shows a detailed schematic view of the system according to the present invention in the example of application of the method of FIG. 1,
FIG. 3 schematically shows an example of application of the method of FIGS. 1 and 2 using the relative system.

### DETAILED DESCRIPTION

The present invention relates to a method and a system 1 (or simulator) for the improvement of a virtual surgical procedure (more briefly hereinafter, surgical procedure), which are applicable in the field of cranial Neurosurgery, for example craniotomy, which represents the first approach for intracranial lesions. In particular, the method and the system 1 of the present invention are applicable for brain tumours, aneurysms, arteriovenous malformations, subdural empyemas, subdural haematomas, intracerebral haematomas. Optionally, the method and the system 1 of the present invention are further applicable for ventricular drainage, ventricular shunt, shunt, and operations that comprise the insertion of a catheter into the ventricular system.

It should be specified here that the present invention relates to a method that allows to improve a cranial surgical procedure, both in the training step of the surgeon without the patient and in the preoperative and operative step, by displaying in augmented reality three-dimensional models of parts of the patient's body that the surgeon can visualize while performing the surgical procedure to perfect his or her technique and in the training and preoperative step, and optionally during an actual surgery. However, although helpful, the method of the invention does not interfere with the normal practice of the surgical procedure which is performed solely and directly by the surgeon.

Preferably, the method for the improvement of a cranial surgical procedure comprises a preliminary step of training an artificial intelligence algorithm A residing in a processing unit 2 to identify a target T that includes the segmentation of the ventricular system from which the identification of the Foramen of Monro, target of said surgical procedure, derives. Preferably, the artificial intelligence algorithm A comprises a convolutional neural network.

The method for the improvement of a cranial surgical procedure comprises the further step of acquiring by means of the processing unit 2 preoperative clinical images of a portion of a patient's body, and in particular of at least a portion of the patient's skull.

The method for the improvement of a cranial surgical procedure comprises the step of generating, by means of the artificial intelligence algorithm A, a three-dimensional holographic model O of the portion of the patient's body, or specifically of the skull, by processing the acquired clinical images. Preferably, this step comprises the step of generating by means of an automatic segmentation algorithm the three-dimensional holographic model O by processing the acquired clinical images.

The method for the improvement of a cranial surgical procedure further comprises the step of identifying by means of the artificial intelligence algorithm A the target T of the surgical procedure in the three-dimensional holographic model O.

The method for the improvement of a cranial surgical procedure comprises the step of acquiring in real time a depth image of the real environment by means of a holographic device associated with a visor 3 for augmented reality wearable by a user and placed in signal communication with the processing unit 2. Preferably, the visor 3 is a visor for mixed reality which, therefore, allows to display holograms projected on a transparent screen so that they are visible by the user wearing the visor 3 having the surrounding real environment as a background. Preferably, the holographic device is a depth sensor of the visor 3, or alternatively, a device distinct from the visor 3.

Still preferably, the method comprises the step of identifying by means of the holographic device a patient in the depth image of the real environment acquired in real time, and more preferably of identifying a specific part of the patient's body such as, for example, the patient's head and face. According to a preferred form of the invention, this step is performed by means of a registration algorithm. Preferably, the registration algorithm resides in the processing unit or, alternatively, in the holographic device.

In addition, the method for the improvement of a cranial surgical procedure comprises the step of displaying on the display of the visor 3 the three-dimensional holographic model O with the target T in overlay to the patient in the real environment, preferably via the registration algorithm.

The method for the improvement of a cranial surgical procedure comprises the step of identifying by means of the holographic device an entry point P of the surgical procedure which together with the target T constitute the surgical trajectory TC for the insertion of the catheter. The pointer 4 that identifies the surgical trajectory TC is movable by the user by interacting directly with the holographic content.

The method for the improvement of a cranial surgical procedure comprises the step of calculating by means of the artificial intelligence algorithm A the target T in the three-dimensional holographic model O which identifies a surgical trajectory TC connecting the entry point P to the target T, i.e. preferably a straight line starting from an entry point up to the centre of the lesion and/or of a surgery site in the skull, passing through the external anatomical layers, such as skin and sub-cutaneous tissues.

According to one aspect of the invention, the identification of the surgical trajectory TC provides for identifying for each point of the intracranial lesion and/or of the intracranial site of intervention the closest point on the skin of the skull through a pre-established algorithm and for joining the centre of mass of the identified points on the skin with the centre of mass of the points of the lesion and/or the site of intervention in order to determine the surgical trajectory TC. Preferably, the pre-established algorithm is of the k-d tree type.

According to the same aspect, in order to perform a cranial surgical procedure such as, for example, a craniotomy or a craniectomy, the method provides for identifying a projection area of the lesion and/or of the intracranial site of intervention on the skull by projecting and connecting the points of the intracranial lesion and/or of the intracranial site of intervention on the skull. Preferably, in order to determine the projection area, a safety margin, for example 1 cm, is considered at the perimeter identified by the projection of the points of the lesion and/or of the site of intervention. Still preferably, the identification of the projection area is carried out by means of a pre-established technique, preferably of the ray tracing type.

The method for the improvement of a cranial surgical procedure also comprises the step of displaying through the display of the visor 3 the surgical trajectory TC together with the three-dimensional holographic model O in overlay to the patient in the real environment. Advantageously, the hologram shows with what inclination to enter and how deep to go in order to reach the target point. Preferably, the automatic registration algorithm overlays the hologram (or three-dimensional holographic model O) of the head with the planning of the surgical trajectory TC to the patient's face by using the information acquired in the depth image.

It should be noted that the method of the present invention therefore allows to display a 3D reconstruction of the internal cranial lesion and/or of the intracranial site of intervention in the hologram together with the surgical trajectory TC and the identified projection area on which to perform a cranial surgical procedure, for example craniotomy. In this way, it is possible to more accurately identify and minimize the length of the skin incision and adapt the size of the craniotomy based on the specific case.

Preferably, the preoperative clinical images of the at least a portion of a patient's skull comprise CT images of the patient's skull. Still preferably, the three-dimensional holographic model O comprises a three-dimensional model of the patient's skin, skull, lateral ventricles and third ventricle.

The system 1 for the improvement of a cranial surgical procedure of the present invention comprises a processing unit 2 and an artificial intelligence algorithm A residing therein and trained, preferably for identifying a target T of said surgical procedure comprising ventricles and Foramen of Monro.

The system 1 comprises a visor 3 for augmented reality wearable by a user and provided with a display for displaying images and with a holographic device for real-time acquisition of a depth image of the real environment. The visor 3 is placed in signal communication with the processing unit 2. As mentioned above, in the preferred form of the invention, the visor 3 is a visor for mixed reality. Preferably, the holographic device is a depth sensor of the visor 3, or alternatively, a device distinct from the visor 3. Preferably, the holographic device is configured to identify a patient in the depth image of the real environment acquired in real time and more preferably to identify a specific part of the patient's body such as, for example, the patient's head and face. Still preferably, the system 1 comprises a registration algorithm configured to identify a patient in the depth image of the real environment acquired in real time. Preferably, the registration algorithm resides in the processing unit 2 or, alternatively, in the holographic device.

The processing unit 2 is configured to acquire preoperative clinical images of a portion of a patient's body, and in particular of at least a portion of the patient's skull. The artificial intelligence algorithm A is configured to generate a three-dimensional holographic model O of the patient's body portion, and specifically of the skull, by processing acquired clinical images, such as for example images obtained by CT.

The artificial intelligence algorithm A is configured to identify the target T of the cranial surgical procedure in the three-dimensional holographic model O.

The display of the visor 3 is configured to display the three-dimensional holographic model O with the target T in overlay to the patient in the real environment, preferably by means of the registration algorithm.

The holographic device is configured to identify in the holographic content a pointer 4 movable by the user on the three-dimensional holographic model O displayed on the display of the visor 3.

Preferably, the pointer 4 comprises an optical pointer configured to emit at least one light signal detectable by the holographic device. Optionally, the pointer 4 comprises one or more markers, either active or passive, configured to reflect and/or emit a light signal so that the holographic device can acquire such signals. Still preferably, the holographic device is configured to calculate the position and/or the orientation of the pointer 4 in space with respect to the global coordinate system.

Alternatively, the pointer 4 comprises an electromagnetic pointer configured to transmit data relating to the position and/or the orientation of the pointer itself with respect to the global coordinate system to the holographic device. Preferably, the pointer 4 is therefore placed in signal communication with the holographic device for receiving and/or transmitting data. Optionally, the electromagnetic pointer may comprise one or more sensors in signal communication with the holographic device.

The holographic device is configured to identify an entry point P of the cranial surgical procedure selected by the user by means of said pointer 4 on the three-dimensional holographic model O displayed on the display.

The artificial intelligence algorithm A is configured to calculate a surgical trajectory TC connecting the entry point P to the target T in the three-dimensional holographic model O.

The display is configured to display the surgical trajectory TC together with the three-dimensional holographic model O in overlay to the patient in the real environment. Advantageously, the hologram shows with what inclination to enter and how deep to go in order to reach the target point T. Preferably, the automatic registration algorithm overlays the hologram (three-dimensional holographic model O) of the head with the planning of the surgical trajectory TC to the patient's face. In this way, as previously reported, it is possible to view a reconstruction of the cranial lesion and/or of the intracranial site of intervention and relative projection area and adapt the surgical procedure of craniotomy to the specific case.

Preferably, the artificial intelligence algorithm A comprises an automatic segmentation algorithm configured to generate the three-dimensional holographic model O by processing the acquired clinical images.

As mentioned above, the registration algorithm is adapted to identify in the depth image of the real environment acquired in real time the anatomical region of interest of the patient, for example head and face, to overlay the holographic model O at the patient. The holographic model O comprises the surgical trajectory identified by the user by means of the holographic device and a pointer 4 movable by the user.

Preferably, the preoperative clinical images of the at least a portion of a patient's skull comprise CT images of the patient's skull. The three-dimensional holographic model O comprises a three-dimensional model of the patient's skin, skull, lateral ventricles and third ventricle.

Preferably, both in the method and in the system 1 usable for its execution, the artificial intelligence algorithm A is configured to recognize other structures from the preoperative clinical images, such as for example blood vessels that can then guide the choice of the entry point P in order to identify non-hazardous surgical trajectories TC.

As anticipated in the present description, the system 1 and the method of the present invention find particular application in neurosurgery in the execution of preoperative planning and assistance in the execution of surgeries through craniectomies or craniotomies with identification of the surgical trajectory TC, of the consequent positioning of the headboard and of the head on the operatory bed, of the consequent site of the surgical incision and finally of the correlated craniectomy or craniotomy.

It should be noted that the system 1 and the method of the present invention also find possible application in neurosurgery in the execution of percutaneous procedures, both cranial and spinal. Some examples of application are listed below:
thermorhizotomies or micro compressions of the Gasserian ganglion, i.e., surgical procedures that are performed with X-ray examination, for the treatment of trigeminal neuralgia;
needle brain biopsies, i.e., surgical procedures guided by the neuronavigator or by the stereotactic helmet;
implantation of deep electrodes for the treatment of movement disorders, i.e., surgical procedures guided by the neuronavigator or by the stereotactic helmet;
infiltration of the intervertebral articular facets, i.e., surgical procedures that are performed with X-ray examination;
positioning of trans-peduncular screws in spinal stabilization surgeries, i.e., surgical procedures that are performed with X-ray examination;
spinal tap.

It should be specified that in the various example of applications, various instruments, not only catheters, can be used to follow the surgical trajectory TC from the entry point (or access point) P on the skin and target T. Advantageously, the method provides a surgical trajectory that can be taken into account by the surgeon and that can allow access to the lesion and/or to the site of intervention, and therefore to the target T, in the most comfortable and least invasive way possible.

Advantageously, by taking advantage of the mixed reality and the automatic registration algorithm, the surgeon can view the internal structures of the patient and see the target T of the surgical procedure highlighted, such as the entry of the foramen of Monro, with eventually the aid of a holographic guide to assist him or her in the insertion. By properly inserting the catheter into the selected entry point (or insertion point) P, the surgeon can proceed by deciding to use the surgical trajectory TC to reach the target T and drain the fluid from the ventricular chambers, reducing the intracranial pressure in situations of need such as hydrocephalus, head trauma and bleeding. It should therefore be noted that the surgical trajectory TC can be considered by the surgeon as a holographic guide to assist him or her in the insertion of the catheter during ventricular drainage operations (or ventricular shunt). However, it should be specified that the surgical trajectory TC is a pure reference. In fact, the surgeon is the only performer of the surgery who can decide to take this trajectory into account in his or her surgery choices. With further detail, the surgeon is able to modify the angle of inclination of the trajectory to avoid neurovascular tissues or structures. As mentioned above, the method and the system 1 of the invention are particularly useful for the practice of surgeons and for use in the preoperative step.

Preferably, the data transmission between processing unit 2 and visor 3 is based on a TCP (Transmission Control Protocol) communication protocol, so as to transfer the patient-specific three-dimensional models to the head-mounted display (or visor 3).

Advantageously, the preferred use of the mixed reality allows the visualization of the structures of interest with the possibility of free interaction with the holograms, which can be used both pre-operatively and intra-operatively.

Advantageously, unlike the known technique, the device and the registration algorithm are of the marker-less type and allow a correct positioning of the holograms on the patient.

It should be noted that, in the example of the accompanying figures, for the lateral ventricles and the third ventricle, the structures are automatically recognized by a specially trained and tested convolutional neural network. Subsequently, these structures are inputted to an algorithm that highlights and saves the entries of the foramen of Monro as an OBJ file, using a criterion based on the distance between the lateral ventricles and third ventricles. The patient-specific 3D model consisting of the OBJ files, and in particular of vertices and faces, of skin, skull, ventricles and foramen of Monro is transferred by means of a TCP communication protocol, based for example on the WebSocket protocol, to the mixed reality visor 3. The latter projects holograms allowing to see the real environment through the display, thus allowing to view the holograms of skin, skull, ventricles, and foramen of Monro. The surgeon can thus interact with the structures in the preoperative step and plan the surgery. In the operative step, the holograms can be positioned on the patient's face using the automatic registration algorithm. The CT images of a patient are processed by algorithms specially created to automatically segment the structures of interest: skin, skull, lateral ventricles and third ventricle. In particular, to obtain the skull, a threshold of 500 HU is applied and an OBJ file is created with a marching cubes algorithm, and in the case of a patient with a dental implant, the region of the mouth is removed using an iterative closest point algorithm. For the skin, a bandpass filter between 100 and 50 HU, morphological opening and closing algorithms to remove the support present in the CT images and Sobel edge detection to extract only the first layer of skin are applied. Also in this case the OBJ file with marching cubes is obtained. The marker-less reconstruction exploits the acquisition of a points (or point cloud) through the depth chamber present in the holographic device, which can also be defined as a registration device, and a registration based on ICP algorithms. The accuracy of the registration was assessed using a 3D printed patient-specific phantom and showed an accuracy of approximately 2.7 mm. By using the method and the system 1 it is therefore possible to display a holographic surgical trajectory TC that acts as a possible trajectory to follow during the insertion of the catheter that connects the Foramen of Monroe to a point chosen by the surgeon on the holographic skin model. Advantageously, the method and the system 1 of the invention allow a considerable saving of the computational cost with consequent reduction of the total estimated time to process a new patient, transfer the files and display the structures in the mixed reality application, which is about 3 minutes.

Below are the main and advantageous improvements given by the method and by the system 1 of the invention compared to the traditional surgical procedure:
the internal structures are allowed to be displayed;
the ideal target of the surgery, the entries of the foramen of Monro are highlighted;
a holographic guide is provided for the correct insertion of the catheter;
there was an improvement in accuracy by 42% in the procedure.

Below are the main advantages given by the method and by the system 1 of the invention compared to the methods and systems for improving a cranial surgical procedure of the prior art:
marker-less registration;
quick automatic segmentation with greater precision;
reduction of the computational times;
an expert operator is not necessary for the segmentation and data transfer steps;
the ideal target of the surgery is highlighted,
manual identification, by means of a pointer, of the entry point of the surgical procedure from which to calculate the trajectory;
mixed and not fully automated procedure, which allows the surgeon a certain autonomy based on experience.

The example of application referred to in the attached FIGS. 1-3 is described in detail below.

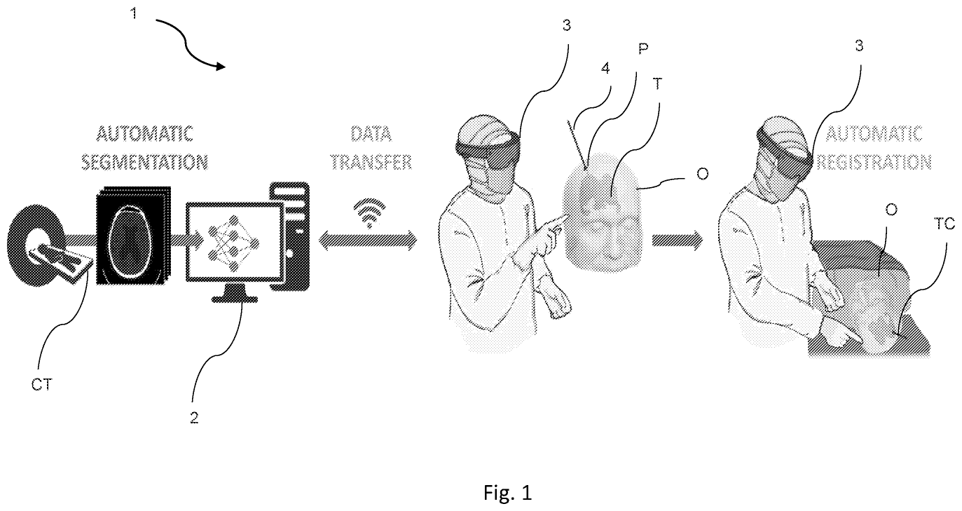

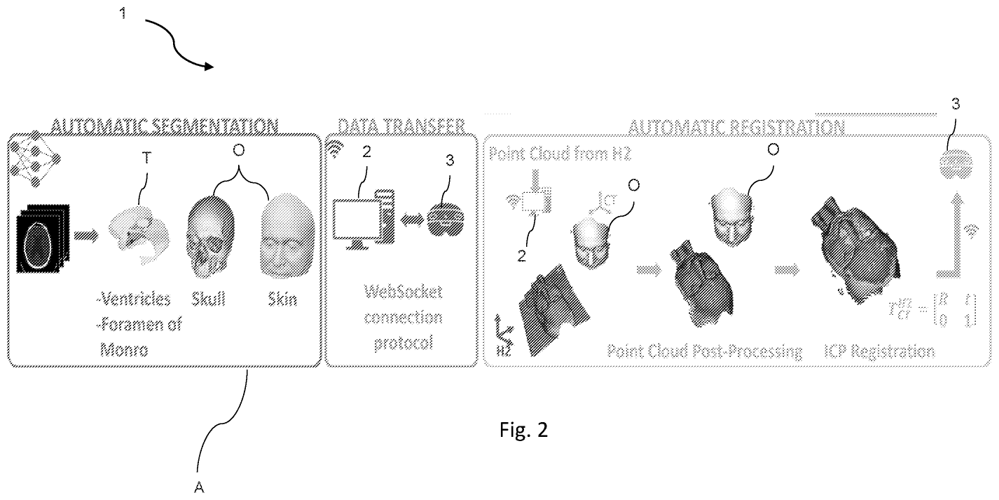

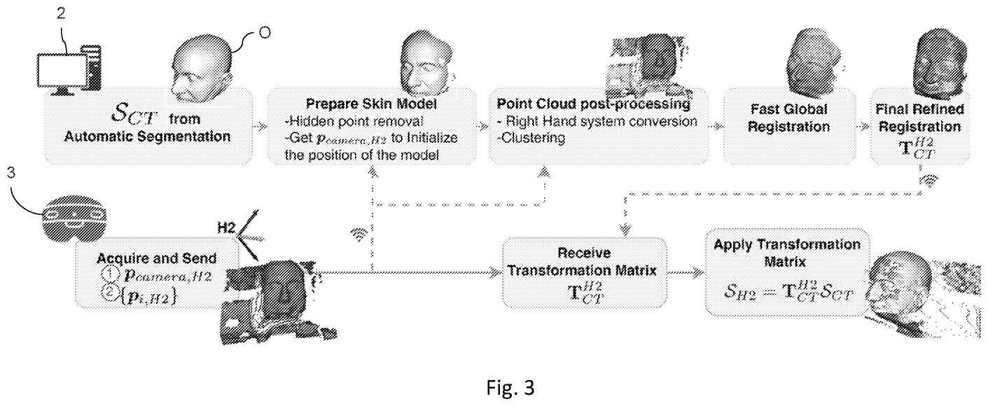

FIGS. 1 and 2 show a schematic representation of the workflow developed according to the system 1 and the method of the invention: the 3D CT (Computerized Tomography) images are automatically segmented to obtain the brain structures that will be sent to the mixed reality visor 3 (indicated in the example of FIG. 3 also with H2) through an Internet connection protocol. The user is thus able to display the holographic model and set a path between the target T, i.e., the Foramen of Monro, and an entry point P on the skin layer. The point cloud PtC of the patient's skin surface is acquired using the depth chamber of the visor H2 and these data are used to estimate the transformation matrix T^{H2}CT.

FIG. 3 depicts in detail the example of the registration workflow for the Mixed Reality (or MR) environment. The artificial intelligence algorithm envisages the following steps:
acquiring the PtC of the facial surface {pi, H2}, using the search mode of the visor H2, and the position of the mixed reality device H2 itself (pcamera, H2), both referring to the global coordinate reference system H2;
initializing the position of S_{CT} with respect to target {pᵢ, H2} based on camera, H2;
applying a hidden point removal algorithm to S_{CT} to filter the exit points on the back of the head to avoid unnecessary calculations;
extracting {pi, H2} points belonging to the face through a clustering algorithm based on a density DBSCAN;
applying a fast global registration algorithm between the simplified S_{CT} and the clean {pi, H2}, obtaining a first alignment, which is then refined through the Local Refined Registration method based on a Point-to-Plane iteration algorithm of the nearest point (ICP) to obtain T^{H2}CT,
sending T^{H2}CT again on H2 and applying to the holographic model of S_{CT}, thus allowing
the surgeon to display the segmented model aligned with the real patient's face: S^{H2}=T^{H2}CT S_{CT}.

1. Method for the improvement of a virtual cranial surgical procedure, comprising the steps of:
acquiring by means of a processing unit preoperative clinical images of at least a portion of a patient's skull;
generating by means of an artificial intelligence algorithm a three-dimensional holographic model of the at least a portion of the patient's skull by processing the acquired clinical images;
identifying by means of the artificial intelligence algorithm a target of the cranial surgical procedure in the three-dimensional holographic model;
identifying by means of a holographic device a pointer movable by the user;
identifying by means of the holographic device an entry point of the cranial surgical procedure selected by the user by means of said pointer on the three-dimensional holographic model displayed on the display of an augmented reality visor wearable by a user and placed in signal communication with the processing unit;
calculating a surgical trajectory connecting the entry point to the target, identified by means of the artificial intelligence algorithm, in the three-dimensional holographic model;
acquiring in real time a depth image of the real environment by means of a holographic device associated with the visor; and
displaying through the display of the visor the surgical trajectory together with the three-dimensional holographic model overlaid to a patient in the real environment.

2. Method according to claim 1, wherein the step of generating by means of the artificial intelligence algorithm a three-dimensional holographic model of the at least one portion of the patient's skull by processing the acquired clinical images, comprises the sub-step of:
generating by means of an automatic segmentation algorithm the three-dimensional holographic model by processing the acquired clinical images.

3. Method according to claim 1, comprising the steps of:
identifying by means of the holographic device a pointer movable by the user in the three-dimensional holographic model and of
identifying by means of the holographic device an entry point of the cranial surgical procedure selected by the user by means of said pointer on the three-dimensional holographic model displayed on the display;

4. Method according to claim 1 claims, wherein:
the preoperative clinical images of at least a portion of a patient's skull comprise CT images of the patient's skull;
the three-dimensional holographic model comprises a three-dimensional model of the patient's skin, skull, lateral ventricles and third ventricle.

5. Method according to claim 1 wherein:
the visor is a visor for mixed reality.

6. System for improvement of a virtual cranial surgical procedure, comprising:
a processing unit and an artificial intelligence algorithm residing therein;
an augmented reality visor wearable by a user and provided with a display for displaying images and with a holographic device for real-time acquisition of a depth image of the real environment, the visor being in signal communication with the processing unit;
wherein
the processing unit is configured to acquire preoperative clinical images of at least a portion of a patient's skull;
the artificial intelligence algorithm is configured to generate a three-dimensional holographic model of the at least a portion of the patient's skull by processing the acquired clinical images;
the artificial intelligence algorithm is configured to identify the target of the cranial surgical procedure in the three-dimensional holographic model;
the display of the visor is configured to display the three-dimensional holographic model with the target in overlay to a patient in the real environment;
the holographic device is configured to identify in the depth image of the real environment acquired in real time a specific part of the patient's body;
the holographic device is configured to overlay the three-dimensional holographic model on the patient in the real environment and to display said three-dimensional holographic model on the display;
the artificial intelligence algorithm is configured to calculate a surgical trajectory connecting the entry point to the target in the three-dimensional holographic model;
the display is configured to display the surgical trajectory together with the three-dimensional holographic model in overlay to the patient in the real environment.

7. System according to claim 6, wherein the artificial intelligence algorithm comprises an automatic segmentation algorithm configured to generate the three-dimensional holographic model by processing the acquired clinical images.

8. System according to claim 6, comprising a registration algorithm adapted to identify by means of the holographic device a specific part of the patient's body and to overlay thereon the three-dimensional holographic model, and a pointer movable by the user in the three-dimensional holographic environment adapted to identify an entry point of the cranial surgical procedure selected by the user by means of said pointer on the three-dimensional holographic model displayed on the display.

9. System according to claim 6,
wherein:
the preoperative clinical images of the at least a portion of a patient's skull comprise CT images of the patient's skull;
the three-dimensional holographic model comprises a three-dimensional model of the patient's skin, skull, lateral ventricles and third ventricle.

10. System according to claim 6
wherein:
the visor is a visor for mixed reality.
